(19) United States
(12) Patent Application Publication
(10) Pub. No.: US 2018/0279939 A1
(43) Pub. Date: Oct. 4, 2018

(54) METHOD AND SYSTEM FOR LOCATING SEIZURE FOCUS FROM INTERICTAL DATA

(71) Applicant: The Children's Medical Center Corporation, Boston, MA (US)

(72) Inventors: Joseph R. Madsen, Boston, MA (US); Eun-Hyoung Park, Boston, MA (US)

(73) Assignee: The Children's Medical Center Corporation, Boston, MA (US)

(21) Appl. No.: 15/735,039

(22) PCT Filed: Jun. 8, 2016
(86) PCT No.: PCT/US16/36462

§ 371 Date: Dec. 8, 2017

Related U.S. Application Data
(60) Provisional application No. 62/172,871, filed on Jun. 9, 2015.

(51) Int. Cl. A61B 5/00; A61B 34/32; A61B 5/0476

(52) U.S. Cl. CPC A61B 5/4094; A61B 5/0476; A61B 34/32

(57) ABSTRACT

Methods and systems for identifying epileptogenic regions of the brain and locating seizure foci can use only interictal EEG data. These methods and systems enable a physician to locate seizure foci without having to wait for a patient to experience a seizure and have it recorded. A network causality analysis can be applied to the interictal EEG data to determine the causal nodes, the nodes influenced by the causal nodes and the causal connection or link between the nodes. The most influential causal nodes can be ranked and used to generate a map of causal links in the brain networks to accelerate the identification of the epileptogenic regions of the brain and the location seizure focus.

### CROSS-REFERENCE TO RELATED APPLICATIONS

This application claims benefit under 35 U.S.C. § 119(e) of the U.S. Provisional Application No. 62/172,871, filed Jun. 9, 2015, the contents of which are incorporated herein by reference in their entirety.

### STATEMENT REGARDING FEDERALLY SPONSORED RESEARCH

This invention was made with US Government support under contract 5R01NS069696-02 awarded by the US National Institutes of Health. The US Government may have certain rights in this invention.

### REFERENCE TO MICROFICHE APPENDIX

Not Applicable

### BACKGROUND

### Technical Field of the Invention

The present invention is directed to methods and systems for locating epileptogenic foci for treatment of epilepsy. More specifically, the present invention is directed to locating the causal part of the epileptogenic network using interictal data to facilitate treatment.

### Description of the Prior Art

Currently, surgical resection is the only curative treatment for patients with medically refractory epilepsy. For successful surgery, the region of the brain which causes seizures must be removed. The challenge of epilepsy surgery is to identify and safely remove the epileptogenic foci or causal part of an epileptogenic network. To accomplish this goal, intracranial EEG (iEEG) recording is frequently used. It is widely accepted in clinical community that interictal EEG data does not allow adequate mapping of the seizure focus, so under the currently accepted practice, iEEG data recorded during seizure events is required in order to identify the seizure onset zone. Therefore, iEEG data is continuously recorded for up to a week while epileptologists wait to observe seizures. Once recorded, they visually analyze ictal iEEG data signals. Under this standard of care, two craniotomies, and prolonged expensive and potentially risky hospitalization of patients are required. In addition to being risky and costly, this approach does not actually reveal the network connectivity.

### SUMMARY

The present invention is directed to methods and systems for identifying epileptogenic regions of the brain and for locating seizure foci using intracranial EEG (iEEG) recordings made during the interictal state, the interval between seizures. The methods and systems according to the invention enable a physician to locate seizure foci without having to wait for a patient to experience a seizure and have it recorded. In accordance with embodiments of the invention, interictal iEEG data can be used to generate a map of causal links in the brain networks to accelerate the identification of the epileptogenic regions of the brain and the location of seizure focus for treatment.

The brain networks supporting seizure generation are present regardless of whether a seizure is happening at any given moment. According to the embodiments of the invention, the causal part of an epileptogenic network to be identified and resected can be detected using intracranial EEG (iEEG) recordings during interictal state.

In accordance with some embodiments of the invention, interictal EEG signals can be monitored and recorded. The interictal EEG signals represent EEG signals detected at predefined locations (e.g., using an array of sensors) in the brain. The recorded interictal EEG signals can be digitized and converted to interictal EEG data. The interictal EEG data can be filtered and/or converted to a predefined data format processing and analysis. The formatted interictal EEG data can be processed using a causality analysis to identify one or more causality links in the network of predefined locations or nodes of the brain. The most influential causal nodes in the network indicate the epileptogenic regions of the brain and the location of the seizure focus for treatment. The nodes and causality links can be mapped to diagram or image of the brain to identify the location for resection. The node information and causality links can be used to control a tool or device that assists the surgeon in identifying the location of one or more seizure foci. The tool or device can be incorporated in the array of sensors used to monitor EEG signals and display elements (e.g. LEDs) to help illuminate the location of one or more seizure foci for resection. In accordance with some embodiments, the node information and causality links can be used to control another system, such as a laser or robot that assists with identifying the location of one or more seizure foci as well as assists with the resection of that portion of the brain.

In accordance with some embodiments of the invention, the method includes surgery, such as a craniotomy and the subdural placement of a grid or array of electrodes on the surface of brain. The intracranial EEG (iEEG) signals detected by the electrodes can be recorded over a predefined period of time. The stored iEEG signal data can be filtered and/or converted to a predefined data format. The formatted iEEG data can be processed using a causality analysis (e.g., Granger Causality) to identify the most influential causal nodes in the sensed brain network. The locations of these nodes correspond to the locations of the electrodes in the grid or array of electrodes. The identity and location of the most influential causal nodes can be used to map the location of seizure foci and control a tool that highlights the location on the brain as well as assist in the resection of that portion of the brain.

In accordance with some embodiments of the invention, the location of the seizure foci can be overlaid on a CT scan image or other three dimensional (3D) representation of the brain. In accordance with some embodiments of the invention, the grid or array of electrodes can include a plurality of lights (e.g., LEDs) or other optical or graphical image that can be controlled to illuminate the location of seizure foci enabling the surgeon to determine the area for resection. In accordance with some embodiments of the invention, the system can include a laser that can illuminate the location of seizure foci as a function of the information about the identity and location of the most influential causal nodes. In accordance with some embodiments, the laser can be used to resect a portion of the brain tissue in the region at the location of the most influential causal nodes.

The methods and systems according to the invention can be used to identify locations in the brain that cause seizures without using EEG data that includes a seizure event. This enables seizure foci to be more quickly identified and located without the need to wait for an EEG recording of a seizure event. The methods and systems according to the invention enable a much shorter procedure and reduced risk and cost to the patient.

These and other capabilities of the invention, along with the invention itself, will be more fully understood after a review of the following figures, detailed description, and claims.

### BRIEF DESCRIPTION OF THE FIGURES

FIG. 1 shows a block diagram of a method for identifying and locating seizure focus using interictal EEG data according to some embodiments of the invention.

FIG. 2 shows a block diagram of a method for identifying and locating seizure focus using interictal EEG data according to some embodiments of the invention.

FIG. 3 shows a diagrammatic view of the grid placement procedure of Block 1 of FIG. 2, according to some embodiments of the invention.

FIG. 4 shows a diagrammatic view of the data monitoring and recording procedure of Blocks 2 and 3 of FIG. 2, according to some embodiments of the invention.

FIG. 5 shows a diagrammatic view of the procedure of Block 4 of FIG. 2, according to some embodiments of the invention.

FIG. 6 shows a diagrammatic view of an example of grid placement and the results of interictal EEG data analysis used to identify the most influential nodes and seizure focus.

FIG. 7 shows a comparison of data from sample patients showing grid placement, connectivity analysis, and causal node identification according to embodiments of the present invention with seizure onset zone information determined clinically through seizure observation.

FIG. 8A shows the mean distance (over 25 patients' cases) from the causal node set to the epileptogenic zone (EZ) node set as a function of the number of electrodes that are included in “high causality set.” The blue dots (lower) indicate mean distance from the causal nodes set to the EZ and the black dots (upper) indicate the mean distance expected by chance.

FIG. 8B shows a Box-Whisker plot of p values (the red horizontal line indicates median value of the total 25 p values) for each top N electrode case (N=1, 2, . . . , 10).

FIG. 9A shows the mean distance (over 25 patients' cases) from the causal node set to the EZ node set as a function of the threshold value. The blue dots (lower) indicate mean distance from the causal nodes set to the EZ and the black dots (upper) indicate the mean distance expected by chance.

FIG. 9B shows a Box-Whisker plot of p values (the red horizontal line indicates median value of total 25 p values) for normalized threshold value (with interval 0.1).

### DETAILED DESCRIPTION OF PREFERRED EMBODIMENTS

The present invention is directed to methods and systems for identifying epileptogenic regions of the brain and locating seizure foci from interictal EEG data, such as intracranial EEG (iEEG or invasive EEG) recordings and/or scalp EEG (or non-invasive EEG) recordings, without the need to record a seizure event. In accordance with some embodiments of the invention, interictal EEG data can be used to generate a map of causal links in the brain networks to facilitate the identification of the epileptogenic regions of the brain and the location seizure focus for resection in a significantly shorter amount of time with less risk to the patient.

In accordance with some embodiments of the invention, the method can include recording EEG signals of the brain during an interictal state to produce interictal EEG data. The interictal EEG data represents the interictal signals detected at predefined node locations in the brain. Using a causality analysis, the most influential causal nodes can be identified as the causal region for seizures. The locations of these most influential causal nodes can be used to determine the location for resection. And the resection can be performed without having observed or recorded the patient having a seizure.

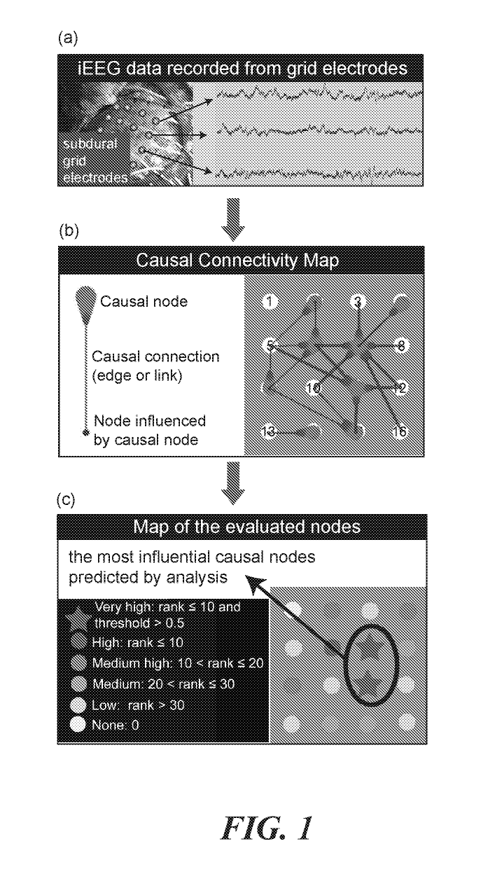

FIG. 1 shows a diagrammatic view of a method according to some embodiments of the invention. And while this embodiment uses iEEG (intracranial EEG), the method can use non-invasive scalp EEG signals, instead of or in addition to iEEG signals. At (a), interictal iEEG data obtained from subdural grid electrodes can be monitored and recorded. At (b), causality analysis can be used to construct a causal connectivity map that identifies causal nodes, causal connections or links, and nodes influenced by causal nodes. At (c), the most influential nodes can be determined by the analysis and a ranking of the most influential nodes can be used to identify and locate seizure focus for treatment. In accordance with some embodiments, the non-invasive, scalp EEG signals can be used to identity target locations of seizure focus for resection according to the methods disclosed herein and then invasive, subdural EEG electrodes can be used to confirm and/or refine the target locations prior to resection.

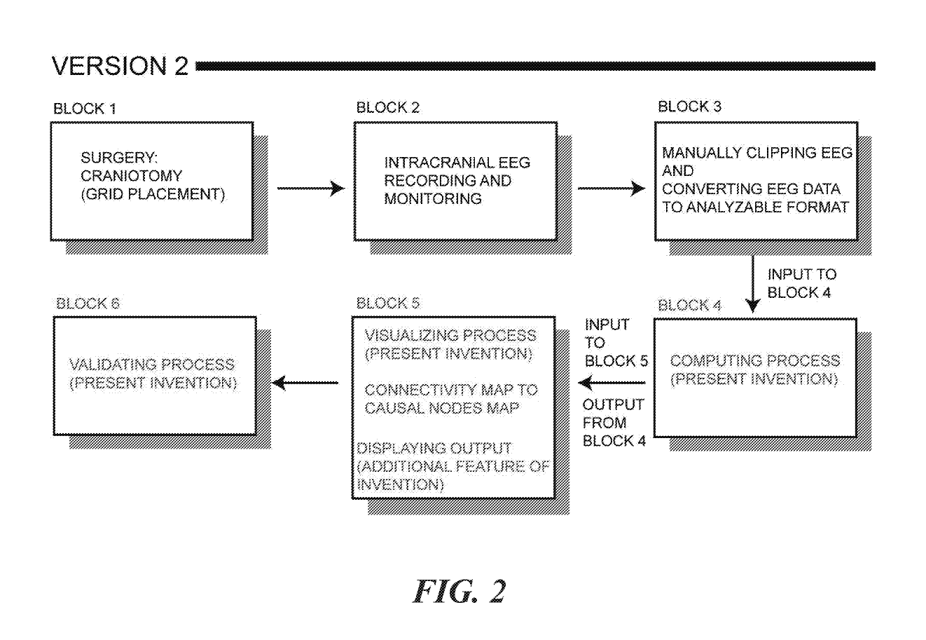

FIG. 2 shows a diagram of a method for identifying and locating epileptogenic regions of the brain according an illustrative example of the invention. This embodiment of the invention includes surgery to expose a portion of the brain to enable the placement of a monitoring array of sensing electrodes (Block 1), monitoring the interictal EEG signals and recording or storing digital data representative of the monitored EEG signals, (Block 2), dividing the EEG signal data in to segments or blocks and converting the raw EEG signal data into a predefined format (Block 3), analyzing the formatted EEG signal data to determine causal nodes (Block 4), constructing the causal network and connectivity between the causal nodes (Block 5), and displaying the causal network and identifying the most influential causal nodes (Block 6). Additional features can include controlling a system to identify the location of the most influential causal nodes, such as using illumination or a laser, and controlling a resection system, such as a robotic surgical system or a surgical laser that can be used to perform the resection.

At Block 1, a craniotomy can be performed to expose the portion of the brain believed to contain one or more epileptogenic regions. One or more arrays of electrodes can be placed on the surface of the brain to detect and record iEEG signals. In accordance with some embodiments, the electrodes can include probes that extend below the surface of the brain in order to monitor subsurface iEEG signals.

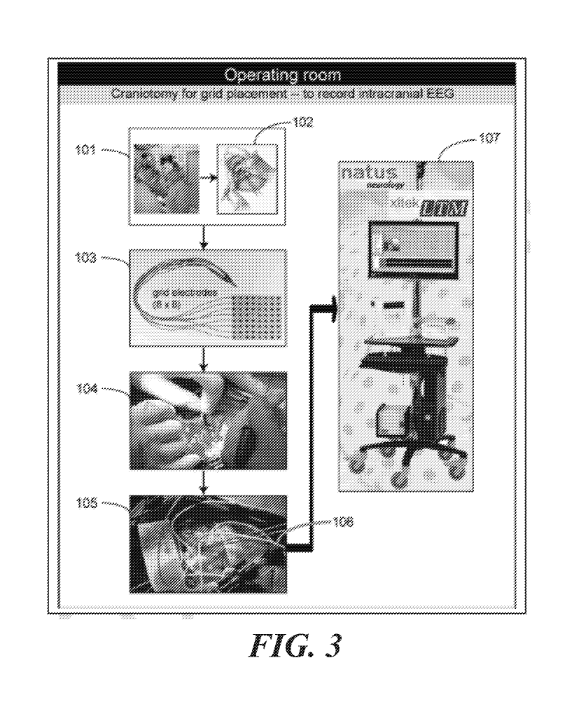

FIG. 3 shows an illustrative diagram of a surgical procedure for grid or array placement. The patient is prepped for surgery and at 101 and 102, a craniotomy is performed. At 103, a grid or array of electrodes is selected. While an 8×8 grid is shown at 103, different sizes of grids or arrays can be used and in some embodiments multiple grids or arrays of electrodes can be used. In accordance with some embodiments, the grid can be one or more 8×8 grids or two or more 8×2 strips of electrodes. At 104 and 105, the grids are placed at predefined locations on the surface of the brain (e.g., subdural electrodes) and below the surface of brain (e.g., depth electrodes). In accordance with some embodiments of the invention, non-invasive surface electrodes can be used in addition to or instead of the subdural (intracranial) electrodes. At 106, cables can be used to connect the grids to the EEG recording device for monitoring and recording the EEG signals. The monitoring device can be, for example, a Natus Neurology, XLTEC L™ EEG monitoring and recording system.

In accordance with some embodiments, the iEEG data can be recorded with subdural and/or depth electrodes (e.g., Ad-Tech, Racine, Wis.) to accurately identify the epileptogenic area. In accordance with some embodiments, each individual platinum electrode contact can be 4 mm in diameter (numbered side of the electrode) with a 2.3 mm diameter exposed recording surface. In accordance with some embodiments, the depth electrodes can contained ten recording contacts (1 mm in diameter per contact) spaced 0.5 cm-1.0 cm apart and linearly arranged. The total number of electrodes for a patient can range from 16 to 256 or more.

At Block 2, the iEEG signals can be monitored and recorded over a predefined period of time. The period of time can range for less than hour to more than 24 hours. In accordance with some embodiments, the iEEG signals can be monitored for as long as 100 hours. In accordance with some embodiments of the invention, the iEEG signals can be monitored and recorded for 1.0 minute or less (e.g., 5 seconds, 10 seconds, 15 seconds, 20 seconds, 30 seconds, or 45 seconds). In accordance with some embodiments of the invention, the iEEG signals can be monitored for the minimum period of time sufficient to identify the seizure focus according to the algorithms used to process the data.

In accordance with some embodiments, the electrical signals from each recording site or node (e.g., channel or variable) can be digitally recorded at sampling rates in the range from 100 Hz to 10,000 Hz, and stored using XLTEK NeuroWorks software (XLTEK, Oakville, Ontario, Canada) and a Natus Database of Natus Neurology (Natus, San Carlos, Calif.).

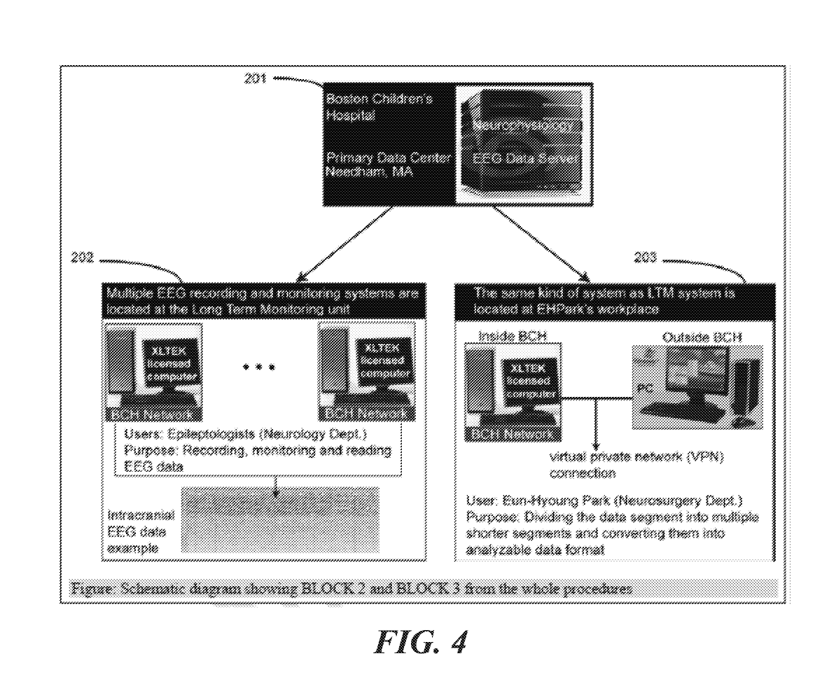

FIG. 4 shows an illustrative example of a system for storing the EEG signal data. After the grids, strips and/or depth electrodes are implanted, the recordings can be started and the data can be stored in a primary server 201. While the data is being recorded, the EEG data can be monitored by epileptologists at a monitoring facility 202. In accordance with some embodiments, to prepare the EEG data for analysis (Block 4), the data segments, which can be 100 hours or more of data, can be cut into shorter EEG data segments.

At Block 3, after the monitoring is completed, the iEEG signal data can divided into segments for formatting and analysis. In accordance with some embodiments, to prepare the EEG data for analysis (in Block 4), the data segments, which can be 100 hours or more of data, can be cut into shorter EEG data segments. The data segments can range from 5 seconds to 20 hours in length. The data segments can be stored in a database for subsequent analysis. In accordance with some embodiments, the iEEG data segments can be converted to predefined data format, such as, European Data Format (EDF) from the XLTEK system and the data can be down-sampled to 200 Hz. In some embodiments, only a twenty-minute interval or segment of interictal data is needed to perform the causality analysis.

In accordance with some embodiments, the recordings from the nodes or channels with obvious noise (such as out-of-range or no signal) can be excluded. Following the noise rejection process, the data sets can be re-referenced to the common average [15, 16, 17]. To achieve covariance stationarity of the data segment, several preprocessing steps such as detrending, demeaning and first order differencing can be used [18, 19, 20, 21]. The nonstationarity in the mean and standard deviation can be removed by subtracting the mean from the data segment and dividing it by standard deviation [18, 20].

At Block 4, one or more of the interictal iEEG data segments can be analyzed to identify the most influential causal nodes using a causal connectivity analysis such as Granger causality. The causality analysis uses the interictal iEEG data segment for each electrode to evaluate the causal influence of each node on the other nodes and to identify the most influential nodes.

In accordance with some embodiments, the causal connectivity analysis can be implemented using the Granger Causal Connectivity Analysis toolbox (version 2.9) in Matlab (available online: http://www.anilseth.com and http://www.sussex.ac.uk/sackler/mvgc/). Granger causality (GC) method is a statistical approach to detect causal influences and evaluate the strength of the causal interactions among simultaneously recorded signals, such as iEEG. It is based on linear regression modeling. The Granger causality method can be used to (statistically) detect a causal relationship between two time series (A and B) of the network using this method: one time series A causes (or “Granger-causes”) another time series B, if the past values of A help to predict the further values of B. In accordance with some embodiments of the invention, where iEEG data are recorded from many channels, multivariate (conditional) Granger causality can be used to evaluate causal influence from one channel while all others are included in the model. The details of the conditional GC are discussed further in the literature [13, 14, 20, 22] which are herein incorporated by reference.

In accordance with some embodiments of the invention, formulating a multivariate autoregressive (MVAR) model can include a process for selecting a model order which refers to how many previous observations are included in the model and are used to predict the present observation. Choosing a model order that is too small can lead to insufficient representation of the data while choosing a model order that is too large can exaggerate minor fluctuations in the data and result in a computationally expensive analysis [20, 23, 24]. To select an optimal model order, several well-known criteria can be used [25]. One set of criteria for selecting an optimal model order is Akaike's Information Criterion (AIC) [26]. In practice, however, it is known that the optimal order can vary depending on criteria used [27, 28, 29]. Several factors, such as, sampling rate and length of the data segment impact the optimal order estimation [27, 28, 29]. In accordance with some embodiments, the model order can be empirically determined using the AIC method as an initial guide to plausible orders [27]. For example, in accordance with some embodiments of the invention, the model order can range from minimum of two to a maximum of 200 [10]. In one study, the averaged model order over all cases was five which corresponds to 25 ms time lag in that study.

In accordance with some embodiments of the invention, to validate the model, a Bonferroni corrected Durbin-Watson test can be performed to determine whether the residuals are uncorrelated [30]. The adjusted sum-square-error of the regression can also be used to evaluate the models and confirm that the mean for all cases was adequately high (e.g., mean is greater than 0.28). In accordance with some embodiments, only significant causal links (e.g., at the p<0.01 level; Bonferroni-corrected F-test) can be selected for further causal connectivity analysis and evaluation of each node in the network [20].

At Block 5, a visual representation of the causality connectivity analysis can be generated to identify the causal nodes. During this process, the highest ranking nodes in terms of causality can be identified and ranked.

At Block 6, the system can display the causality map showing the location of the most influential nodes to assist the clinical team to identify one or more locations of the brain for resection.

In accordance with some embodiments, the computed causal connectivity can be visualized on a schematic map of grid locations on the brain (see FIG. 1, elements b and c). As shown in the schematic diagram, causal connections and nodes can be visualized together. In accordance with some embodiments, the network can be characterized by evaluating each node (or electrode), for example by estimating the sum of strength of all the connections leading from a single node to other nodes. In the graph theory terminology, this sum of strength is termed the weighted out-degree [30]. This measure can be normalized by its maximum value to scale the values between zero and one. In the schematic map, the causal nodes can be color-coded to indicate rank ordering of the summed value to distinguish the most influential causal nodes from the least influential nodes.

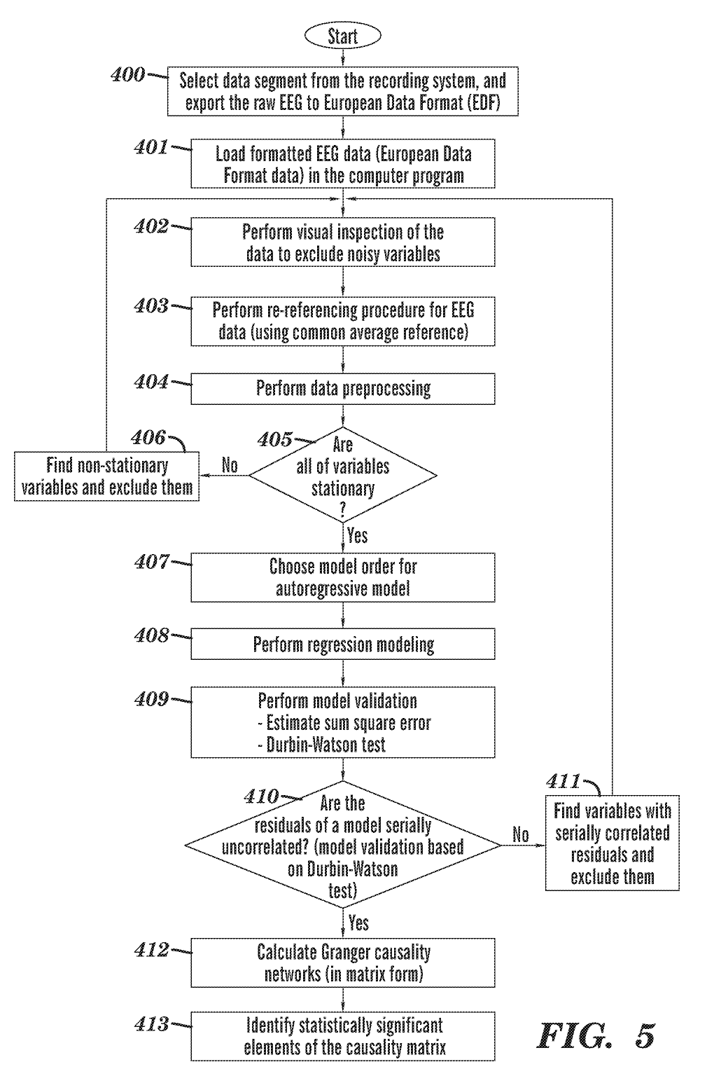

FIG. 5 shows a flow chart of a process for performing a causality analysis according to some embodiments of the present invention. At step 400, the data segment for analysis is selected from the recording system and exported into a predefined format. At step 401, the formatted EEG data can be loaded into the analysis system. At step 402, the data can be inspected to remove noisy channels (e.g., out of range or no signal). At step 403, a re-referencing process can be used to re-reference the EEG data to a common average. At step 404, the data can be pre-processed. At step 405, the method tests whether all the variables (e.g., channels) are stationary and if not, the non-stationary variables are excluded at step 406 and the process returns to step 402. If all the variables are stationary, the process proceeds to step 407 where a model order is chosen. At step 408, regression modeling is performed. At step 409, the model is validated (e.g., estimate the sum square error and perform Durbin-Watson test). At step 410, the method tests whether the residuals of the model are serially uncorrelated based on the Durbin-Watson test. If not, the variables (or electrodes or channels) that have serially correlated residuals are excluded at step 411 and the process returns to step 402. If the residuals of the model are serially uncorrelated the process proceeds to step 412. At step 412, the Granger Causality Network is determined and at step 413, the statistically significant interaction (or links) between nodes in the causality network are identified. Each of these steps is described in further detail below.

As shown in FIG. 5, the process begins with the selection of a data segment, 400. In accordance with some embodiments of the invention, the EEG data (e.g., intracranial (invasive) EEG data and/or scalp (non-invasive) EEG data) can be recorded using an XLTEK system (Natus Neurology Incorporated), a portable type of the system that can be used in the operating room, as shown in FIG. 3. The data segment (e.g., raw EEG data) can be selected and clipped using the XLTEK system or another system and then converted to a computationally analyzable or compatible data format such as European Data Format (EDF) (e.g., using the XLTEK system or another system).

In accordance with some embodiments of the invention, the data segment can be selected from data collected on the same day of the subdural grid and/or depth electrodes placement or within one or two days from the point of placement, as this facilitates rapid visualization of the causal network using interictal data and provides for “quiet” interictal data segment before any seizure events occur.

In accordance with some embodiments of the invention, the interictal data segments can be randomly selected from the intracranial EEG recordings early in the invasive monitoring process for an epilepsy patient who is committed to have resective surgery and is undergoing invasive monitoring.

In accordance with some embodiments of the invention, the segment can be selected to avoid bias and/or noise by taking into consideration the annotation information provided by EEG technologists during recording process. For example, the data in the recording system (e.g., the XLTEK system) can be reviewed for annotations made by EEG technologists (e.g., the time-frames of the “samples” are annotated and annotations can be used to identify quiet interictal state where no abnormal activity was observed by the clinician). Where no abnormal event or seizures are observed, this “sample” period can be considered an interictal (i.e. “between seizures”) state. In accordance with some embodiments of the invention, the earliest available segment of the “sample” period (e.g., before the first seizure happened) can be selected and clipped. In accordance with some embodiments, to remove the effect of transients, some of the data at the beginning and/or the end of the segment can be removed as described herein.

In accordance with some embodiments, the segment suitable for the analysis can be selected to avoid noise and/or external influences. For example, where the annotations, indicate that “patient is talking to nurse” or “Dr. xyz come into the room”, or “xyz channels need to be reconnected,” these notes identify sample periods that can be avoided as the data recorded during those periods may not be suitable for analysis of causal connectivity since it is possible that the noise or external influence could have corrupted the recorded signals.

In accordance with some embodiments of the invention, where the patient is seizing frequently, the earliest interictal segment that does not suggest the influence of noise or external influences can be selected.

In accordance with some embodiments of the invention, the data segment can be chosen from one or more “ictal” data segment(s) (e.g., the intracranial EEG (iEEG) or scalp EEG data segment during seizure events). In accordance with some embodiments of the invention, the ictal segments for some or all seizures that have occurred during the monitoring period. For long-term monitoring periods, patients can have multiple seizures (from one to more than 20 times and sometimes more than 50 times).

In accordance with some embodiments of the invention, the data segment can be chosen from pre-ictal intracranial EEG data, where “pre-ictal” can be a predefined timeframe with respect to seizure onset time. For example, the timeframes of the pre-ictal segment can be 2-42 s before the visible electrographic (seizure) onset, or 30 s immediately preceding the electrographic seizure onset.

In accordance with some embodiments of the invention, the data segment can be chosen from one or more segments where there is a transition from the pre-ictal to the ictal state. The data segments can include changes in the causal connectivity pattern before and during the seizures.

In accordance with some embodiments of the invention, the data segment can be chosen from one or more segments where there is a transition from interictal through pre-ictal to ictal.

In accordance with some embodiments of the invention, the data segment can be chosen from one or more segments where the interictal segment was recorded 1 h before the first seizure.

In accordance with some embodiments of the invention, the data segment can be chosen from two or more segments recorded at least two hours before each seizure events (e.g., 2, 3, 4, 5, 6 or more hours before seizure events). In accordance with some embodiments of the invention, the data segment can be chosen from one or more segments recorded any time (e.g., any number of hours, minutes, seconds or portions thereof) before each seizure.

In accordance with some embodiments of the invention, the size (e.g., time duration) of the selected data segment or segments can range from less than 5 seconds to 1 hour or more. The selection of the size of the duration period can be a function of the compromise between speed and processing efficiency recognizing that a longer duration period providing more data can provide more information to obtain a better outcome at the expense of longer processing times that could increase the risk to the patient in terms of delay and the recognition that as the selected duration period get larger, the benefit of processing additional data becomes diminished. In addition, as computer processing performance continue to improve, larger duration periods can be processed in less time.

In accordance with some embodiments of the invention, the raw EEG data can be converted to a predefined format (e.g., European Data Format, EDF or EDF+) for input into the computer system for computational analysis at 401. EDF (European Data Format) is an example of a standard format for exchange and storage of (bio)physical signals. The signals can have any (and different) physical dimensions and sampling frequencies. EDF was published in 1992 in Electroencephalography and Clinical Neurophysiology, 82: 391-393. EDF+ is a more detailed specification of EDF that is compatible with EDF with some exceptions. EDF+ was published in 2003 in Clinical Neurophysiology 114(9):1755-1761. In accordance with some embodiments of the invention, the EDF formatted data can be converted to numerical data (e.g., ASCII numerical data) and input into the computer system for computational processing.

In accordance with some embodiments of the invention, a signal processing device including an analog to digital converter (ADC) can be used to convert the raw EEG data to a compatible digital data format and input into the computer system for computational processing at 401.

As shown in FIG. 5, at 402, the data can be processed to remove noisy and/or irrelevant variables or channels that may cause spurious and/or contaminated causal connectivity. In some cases, one or more channels may include noise (e.g., the electrode has intermittent contact with the tissue) and these signals are outside the normally expected amplitude and/or frequency range. These noisy signals can be filtered to remove the noisy portions (e.g., in the time domain or the frequency domain) of the signal. Alternatively, the noisy signals can be removed by excluding the channels that include the noisy signals. In accordance with some embodiments of the invention, a computer-based or digital signal processing (DSP)-based algorithm can be used to process the signals to filter or remove the out-of-range signals. In accordance with some embodiments of the invention, the user can visually review the signals and remove or filter the signals of each channel to remove or reduce noise.

In accordance with some embodiments of the invention, the noise can be reduced or removed by using an algorithm, e.g., a factor model combined with principal component analysis (PCA) [42], to select one or more groups (or regions-of-interest) of the electrodes to reduce the number of channels (e.g., electrodes).

In accordance with some embodiments of the invention, the noise can be reduced or removed by excluding channels that show out-of-range signals (e.g., amplitude greater than 1000 microvolts, 1500 microvolts, 2000 microvolts, or other range depending on the characteristics of the electrode and the sensing system). This can be accomplished using a signal processor, a notch filter and/or a low pass filter or by manually selecting the channel for exclusion. In accordance with some embodiments of the invention, the periodic noise (e.g., 50 Hz or 60 Hz power line noise) can be reduced or removed by using a notch filter (e.g., Chebyshev notch filter or a Butterworth notch filter) that blocks 50 Hz or 60 Hz signals.

As shown in FIG. 5, at 403, the EEG signal data can be processed or pre-processed to re-reference the data to deblur the signals and remove the influence of the reference electrode. In some embodiments, each subdural electrode (and/or each depth electrode) is referenced to a single electrode outside of the perisylvian cortex and it may be desirable to remove the influence of the electrode (e.g., make the signal “reference free”). In accordance with some embodiments, the EEG signal can be processed using a Common Average Reference (CAR) spatial filter or a common average reference method to re-reference the electrode. Examples of how to calculate the CAR and Laplacian filter can be found in Crone et al (2001) [15] and “A Practical Guide to Brain—Computer Interfacing with BCI2000” (2010) (section 2.4.2, Spatial filtering) [43].

As shown in FIG. 5, at 404, the EEG signal data can be processed or pre-processed to reduce the sampling rate (resampling) and/or establish covariance stationarity (i.e. the mean and variance of the time series do not vary over time). In accordance with the invention, detrending and demeaning processes can be used to remove non-stationarity in the mean and normalization processes can be used to remove non-stationarity in the standard deviation.

In accordance with some embodiments of the invention, the intracranial EEG signal can be digitally recorded with sampling rates between 250 and 2500 Hz (e.g., using XLTEK NeuroWorks (XLTEK, Oakville, Ontario, Canada) and Natus Database (Natus Neurology, San Carlos, Calif.)). The sampling rate can be different using different equipment and can vary from patient-to-patient. In accordance with some embodiments, the processing time can be reduced by down sampling the EEG signal data to 200 Hz or lower. In accordance with some embodiments, the EEG signal data can down sampled to lowest sampling rate that will enable the process to effectively identify the seizure focus.

In accordance with some embodiments of the invention, the EEG signal data can be processed to remove trend, e.g., the gradual change in the mean or other statistical properties of data. The signal trend can be estimated by fitting a linear function to the data and then the estimated trend can be subtracted from the original time series.

In accordance with some embodiments of the invention, the EEG signal data can be processed to remove the mean be demeaning. For example, the signal mean of the time series for each channel can be computed and then subtracted from the original time series.

In accordance with some embodiments of the invention, the EEG signal data can be processed to normalize the signal. For example, after the demeaning process, the time series can be divided by the temporal standard deviation of the time series to normalize the signal data.

In accordance with some embodiments of the invention, the EEG signal data can be processed to remove the effect of transients. For example, the signal can be processed to remove the first N data points and the last N data points, where N can equal 10, 20, 30, 40, 50, 60, 70, 80, 90, 100, or more data points. Removing transients by removing some of the first and last data points can be used to provide improved statistical significance (when comparing the calculated causal node map with the map of epileptogenic zone manually identified by clinicians).

In accordance with some embodiments of the invention, a non-stationary time series (e.g., intracranial EEG data) can be transformed into a stationary time series by “differencing” the time series. For example, the first difference of the time series x(t) can be expressed as (x(t)−x(t−1)) where t indicates a time point. In accordance with some embodiments, the EEG signal can be processed to do differencing one or more times according to requirements of the system and nature and characteristics of the time series data.

In accordance with some embodiment of the invention, the EEG signal time series data can be processed to determine a region-of-interest (e.g, using PCA or other methods), resulting in not all of channels being used (e.g., a smaller number of channels/variables can be included in the analysis). One or more of the following additional processes can be applied to region-of-interest, including resampling, detrending, demeaning, normalizing and differencing to transform the data into covariance stationary.

In accordance with some embodiment of the invention, the EEG signal time series data can be processed to select a region-of-interest interest (e.g., using PCA or other methods), resulting in not all of channels being used (e.g., a smaller number of channels/variables can be included in the analysis) and then resample the selected region-of-interest time series (e.g., which ranges, normally, from 500 Hz to 2000 Hz for intracranial EEG data) to less than the Nyquist frequency (e.g., half of the original sampling rate). For example, the process can include starting with all electrodes (e.g., channels) then 1) exclude all noisy and unrelated channels and then 2) resampling the remaining channels from their original sampling rate to a lower sampling rate providing a sample time series that can be processed more efficiently to determine Granger Causality. For example, an original time series having a sampling rate of 500 Hz can be down-sampled to 100 Hz or 150 Hz or 200 Hz; an original time series having a sampling rate of 1000 Hz can be down-sampled to 100 or 150 or 200 or 250 or 300 or 350 or 400 or 450 Hz, and an original time series having a sampling rate of 2000 Hz can be down-sampled to 100 or 150 or 200 or 250 or 300 or 350 or 400 or 450 or 500 or 550 or 600 or 650 or 700 or 750 or 800 or 850 or 900 or 950 Hz.

After resampling, one or more of the processing functions can be performed including detrending, demeaning, and/or normalizing. Then, the resulting time series data can be tested using the Kwiatkowski-Phillips-Schmidt-Shin (KPSS) test to determine whether each of the time series or channels is covariance stationary. If any channels do not pass the KPSS test, then the system can process that channel to perform first order differencing. Each channel can be tested using the KPSS test and then processed using first or higher order differencing until the KPSS test is passed. After all the channels pass the KPSS testing and are covariance stationary, the resulting EEG signal data time series can be submitted for Grange Causality determination. In accordance with some embodiments, some channels can be excluded if they cannot be processed to be stationary.

In accordance with some embodiments of the invention, each of the time series data for each channel can be tested using the KPSS test to confirm that each time series is transformed into covariance stationary data. If all the channels pass the KPSS test the data proceeds to 407 where a model is chosen. If one or more channels fail the KPSS testing, the non-stationary channels can be removed or excluded at 406 and the remaining time series data returns to element 402 and repeats elements 403, 404 and 405 until all the channels are stationary. Open Source Software for performing the KPSS Test can include the cca_kpsss.m function that is part of the Granger Causality Connectivity Analysis (GCCA) Toolbox available from http://www.sussex.ac.uk/Users/anils/aks_code.htm and documented at http://www.sussex.ac.uk/Users/lionelb/MVGC/.

As shown in FIG. 5, at 407, after the data is determined to be stationary, the model order can be determined or selected. Granger causality (GC) analysis is a statistical approach to assess causal relationship and evaluate the strength of the causal interactions between time series data (e.g., intracranial EEG data or scalp EEG data) from multiple nodes (e.g., 10, 20, 50 or 100 or more nodes or channels). The GC calculation is based on autoregressive modelling. Formulating the multivariate autoregressive (MVAR) model includes selecting a model order which refers to how many previous observations are included in the model and used to predict the present observation. Choosing too few observations (e.g., a small model order value) may lead to insufficient representation of the data while selecting too many observations (e.g., a large model order value) may exaggerate minor fluctuations in the data and result in a computationally expensive analysis. In accordance with some embodiments, an optimal model order can be determined using Akaike's Information Criterion (AIC). The determined order can vary depending on the criteria used and factors such as the sampling rate, the length of the data segment, and the characteristics of the data can be used to determine the optimal model order.

In accordance with some embodiments of the invention, the model order can be empirically determined using AIC as an initial guide to identify a range of possible orders (e.g., a minimum of 5 and a maximum of 12). The AIC estimation of model order can be determined using, for example, the cca_find_model_order.m function disclosed in the GCCA Toolbox. In accordance with some embodiments of the invention, if the calculated model order is determined to be near the limits or outside of the range, the order can be adjusted to fall with the range or the range can be revised. For example, if the AIC estimates the model order to be 5, the applied model order can be adjusted to be 6 or 7.

In accordance with some embodiments of the invention, process for determining the model order can include using Akaike's information criterion (AIC), Bayesian information criterion (BIC) or Schwarz criterion (SBIC), the minimum description length (MDL) (which is similar to BIC), the final prediction error (FPE), fast orthogonal search (FOS), and/or optimal parameter search (OPS) functions. [44][45] For example, AIC (or BIC) can be used to estimate the model order within the range 4 or 5 to 12. When the sampling rate is 200 Hz, the corresponding time lag ranges 20 ms (=( 1/200 Hz)*4) to 60 ms (=( 1/200 Hz)*12). The maximum model order can be larger than 12 if the computer system is more powerful. In accordance with some embodiments, the model order is selected to be the minimum that enables the most useful Granger Causality calculation for the condition of the patient. Choosing it too small may lead to insufficient representation of the data while selecting it too large may model and exaggerate minor fluctuations in the data and result in a computationally expensive analysis.

In accordance with some embodiments, depending on the sampling rate, one can choose smaller or larger model order whereby the minimum time lag can be at least longer than, for example, 20 ms. The time lag can be determined as a function of the model order and the sampling rate. The model order indicates the number of prior time series data points that are to be used by the model to predict the next data point. Thus, for example, using a sample rate of 200 Hz results in a time interval of 5 ms between data points and for a model order of 5, the lag would be 25 ms. The duration of the minimum time lag can be selected to be greater than the time duration (e.g., the minimum time duration) of the observed iEEG spike for the subject being treated. In accordance with some embodiments, the range of EEG spikes can be between 15 and 80 ms. [46][48].

In accordance with some embodiments of the invention, the model order can be determined as an average of the model order determinations for two or more data segments or channels. For example, each data segment can range from 2 seconds to 2 hours (or longer depending on computation power). After data segmentation is completed, a model order can be determined using the methods described herein for each data segment and then an average model order can be used.

In accordance with some embodiments of the invention, the model order can be selected from a fixed range (e.g., 3-13) and adjusted based on prior computation or empirical experience. The selected model order can also be adjusted up or down, within the range or the range can be adjusted up or down as a function of the sampling rate of the system (e.g., adjusting up for higher sampling rates and adjusting down for lower sampling rates).

As shown in FIG. 5, at 408, after the model order is determined, the regression modeling can begin and the Granger causal relationship matrix can be determined. The Granger causal relationship matrix can, for example, be an N×N matrix, where N indicates the total number of electrodes or channels used to record the intracranial EEG time series data. The Granger causality calculation can be determined using linear regression modelling. In accordance with some embodiments of the invention, Granger causality can be used to assess causal relationships, and to determine and evaluate the strength of the causal interactions between the intracranial EEG time series data. For example, for the causal relationship, we can state that one time series X causes (or “Granger-causes”) another time series Y, if the past values of X help to predict the future values of Y.

In accordance with some embodiments, the Granger causality can be formulated in the time domain using the functions provided in the GCCA Toolbox.

As shown in FIG. 5, at 409, after the Granger Causal relationship matrix is developed, the model can be validated to determine whether the multivariate autoregressive (MVAR) model sufficiently captures the correlation of the structure in the data. In accordance with some embodiments of the invention, the model can be validated by determining the adjusted sum-square-error (RSSadj) of the regression and performing the Durbin-Watson (DW) test or the Bonferroni corrected DW test. This can be accomplished, for example, using the cca_whiteness and cca_granger_regress functions provided in the GCCA Toolbox.

In accordance with some embodiments of the invention, the RSSadj can be used to evaluate whether the amount of variance is sufficiently high for the MVAR model. The RSSadj can vary depending on the system used, but in some embodiments the RSSadj should be at or above approximately 0.3.

In accordance with some embodiments of the invention, the DW or the Bonferroni corrected DW test can be used to evaluate whether the residuals of the MVAR model are serially uncorrelated and provide an indication that the model accurately captures the data structure.

In accordance with some embodiments of the invention, the model can be validated using the Bonferroni corrected Durbin-Watson test to examine whether the residuals are uncorrelated. [30] And the estimated adjusted sum-square-error of the regression can be used to evaluate the models. The threshold can be determined by evaluating the models for multiple cases and then determining an average over all the cases. In accordance with some embodiments, the averaged value (e.g., mean±SD: 0.46±0.18) was considered adequately high. Only significant causal links (e.g., at the p<0.01 level; Bonferroni-corrected F-test) were included for further causal connectivity analysis and evaluation of each node in the network. [20]

As shown in FIG. 5, at 410, the results of the DW test or the corrected DW test are evaluated for each variable or channel. If any of the variables or channels are determined to have serially correlated residuals, those channels are excluded at 411 and the process returns to element 402 and the process is repeated without the excluded variables or channels. If none of the variables or channels are determined to be serially correlated, the process continues to calculate the Granger causality matrix.

As shown in FIG. 5, at 412, the Granger causality matrix is determined. In accordance with some embodiments of the invention, the Granger causality matrix can indicate causal connections from one electrode to another electrode in the form of an N×N matrix where the rows of the matrix indicate the “from” direction or causal nodes and columns of the GC matrix indicate the “to” direction or nodes that are influenced by causal nodes. The cca_granger_regress function of the GCCA toolbox can be used to determine the elements of the GC matrix. The time series data in the form of a matrix (for each node of the Granger causality matrix), along with an estimate of the model order (e.g. 5) and an optional statistical significance test (e.g. F-test) flag can be input into the cca_granger_regress function and for each node, the cca_granger_regress function generates a log ratio causality magnitude (ret.gc), a granger causality probability (reg.prb), a residual sum-square (ret.rss), an adjusted residual sum-square (ret.rss_adj), F-statistics (ret.fs), and autocorrelations in residuals (by Durbin Watson) (ret.waut).

As shown in FIG. 5, at 413, the statistically significant causal connections are identified. The statistically significant causal connections can be identified using the cca_findsignificance function of the GCCA toolbox. For the nodes of the GC matrix, the ret.prb value from cca_granger_regress function, along with a threshold (p value) and a correction flag can be input into cca_findsignificance function. This function returns a matrix (entitle “PR”) that consists of ones and zeros (1s indicate significant interactions by 1s and 0s indicate insignificant interactions) along with the threshold, q, that was applied based on the selected correction (which means that q can be “p value with no correction” when the correction option flag (“CFLAG”) is set to 0, or “p value with Bonferonni correction” when the option flag is set to 1, or “p value with false discovery rate correction” when the option flag is set to 2, or “p value with the approximate false discovery rate correction” when the option flag is set to 3). To obtain “significant” Granger Causality (GC) matrix, the GC matrix (that was computed at the step 412) and the PR matrix (which includes information of the significant connections and the insignificant connections) can be multiplied by using “element-by-element multiplication” in Matlab (syntax: matrix A.*matrix B). [20][22]

In accordance with some embodiments of the invention, the connectivity matrix can be formatted in a graph representing each node as a point or dot with causal nodes having arrows or colored dots or colored lines emanating from them. The graph can be overlaid onto a volume-rendered CT image of the brain to help identify the location of the causal nodes. However, this can sometimes lead to a complex map. In accordance with some embodiments of the invention, the map can be simplified by ranking the strength of the nodes and representing the nodes in different colors according to their causal strength. In accordance with some embodiments of the invention, the map can be simplified by ranking the strength of the causal nodes and hiding or removing the weaker causal nodes (e.g., the nodes that fall below a predetermined threshold).

In accordance with some embodiments, the sum of the strength of the links emanating from each node can be estimated and used to evaluate the causal properties of each node. The sum of the strength can be determined by the weighted out-degree of each node. To evaluate a node (or an electrode) in the causal network, the weighted out-degree can be calculated from the sum of weights (or strengths) of causal links emanating from each node. The weighted out-degree of a node i is defined as follows:

$${k}_{i}^{wout} = {\sum }_{j ∈ {N}_{i}} {w}_{ij} \qquad ( 1 )$$

where Nᵢ indicates the number of neighbors of a node i, and wᵢⱼ is the weight of the link between two nodes (i, j) (wᵢⱼ>0 for j∈Nᵢ; wᵢⱼ=0 for j∉Nᵢ), and the direction of the causal link is from a node i to a node j [30].

In accordance with some embodiments of the invention, the weighted out-degree of each node can be used to rank each according to its causal strength and this ranking can be used to identify the locations of seizure focus. The top 5 or 10 ranked nodes with the highest weighted out-degree can be identified as possible epileptogenic foci for treatment.

In accordance with some embodiments of the invention, the node(s) identified according to the invention as locations of seizure focus can be validated by comparing the identified nodes with nodes identified by clinicians for the same patients. Where there is a high correlation between the nodes identified by the clinicians and the nodes identified according to the invention, the system can be validated.

The top 5 or 10 ranked high causal (HC) area nodes can be compared with the epileptogenic zone (EZ) or the resected zone (RZ) identified by the clinician. The HC nodes can be compared with the clinically identified set of electrodes (the EZ set and the RZ set) by calculating rank order sum. In order to obtain a physically interpretable measure of how well the (HC) set matched the EZ, the distance of each of the high causality (HC) electrodes to the nearest member of the EZ and the RZ can be calculated using an average minimum pairwise distance calculation. The high causality set of electrodes can be identified by assessing the average of mean distances (from the HC set to the EZ set, and from the HC set to the RZ set) over all patients as a function of N_{hc} electrodes (i.e. the number of rank-ordered electrodes included in the HC set). In accordance with some embodiments, a mean distance value of 1.5 cm can be used as a reference value for determining the HC set. In accordance with some embodiments, the top 5 electrodes of the HC set can be the minimum set meeting this condition.

In accordance with some embodiments, the validation can include, for each patient, two or more epileptologists can read intracranial EEG recordings, and identify a set of electrodes as seizure focus. To validate and quantify the correlation or similarity between the clinician determined seizure focus and the high-ranked set of electrodes determined according to the invention (e.g. as highly influential causal nodes), the electrodes can be rank-ordered according to the measure or magnitude of causal influence of each electrode, the ranks of the subset (Nₛ) of electrodes identified by clinicians can be examined and collected, and all ranks can be summed them up. This rank order sum can be used as a statistic to test the null hypothesis that the estimated rank order sum for each patient is not different from what is expected by chance. A Monte Carlo simulation technique can be used to create the sampling distribution of the rank order sum.

The Nₛ number of integers out of total ranks of Nₜ (e.g., which equals the total number of electrodes implanted in each patient) were randomly selected and the values were added together. By repeating this process 100,000 times, the null distribution of rank order sum can be generated. The approximated p value can be computed as the fraction of the 100,000 simulated rank order sum values in the sample distribution that show less than the estimated rank order sum value. This one-tailed test would be proper since a smaller number can be used to indicate a higher rank. To determine whether the results of the 25 individual tests combined are significant under the same null hypothesis, Fisher's method can be applied to compute overall p value and validate the predicted high causality nodes.

In accordance with some embodiments of the invention, the predicted causal nodes can be validated by estimating the minimum pair-wise distance from the predicted causal nodes to the clinically identified nodes. To assess the physical closeness between the high causal nodes and the epileptogenic foci (and to the resected area), an averaged minimum pairwise distance can be estimated by measuring the shortest lengths of distance between each pair of electrodes from the two compared sets. The set of high causal nodes for each patient can be selected based on the threshold of normalized causal strength. Since the distribution of the strength of causal nodes (or the values of weighted out-degree for individual electrodes) can vary for each patient case, the threshold can be adjusted so that the number of electrodes in each set of high causal nodes is more than at least three.

To calculate the distance, CT images can be used to obtain coordinates of implanted electrodes for each patient (e.g., using the medical imaging software, such as 3D Slicer version 4.2.2.1 available from Brigham and Women's Hospital, Boston, Mass. (www.slicer.org). To test the null hypothesis that the estimated distance is not different from what is expected by chance, a null distribution can be created for each case by randomly shuffling the data, selecting electrodes whose strengths of causal influence are greater than the threshold for each patient, and estimating minimum pairwise distance between the randomly sampled set and the electrodes determined clinically. This process can be repeated a large number of times (e.g., 50000 to 100,000) times, which can be dependent on the convergence of the distribution. In some cases, it could also be possible that the convergence can be reached with only 5000 times). Once each expected value (i.e. a distance expected by chance for each case) for each electrode distance is obtained, a Wilcoxon rank sum test can be used to test the statistical significance and validate the prediction.

In accordance with some embodiments of the invention, other types of data can be utilized to locate seizure onset zone. This other data can include magnetic resonance imaging (MM) data, functional MRI (fMRI) data, magnetoencephalography (MEG) data, scalp EEG (or non-invasive EEG) data, transcranial magnetic stimulation (TMS) data, intracranial EEG (invasive EEG) and, as well as combinations thereof. The epilepsy clinical team can assess each patient's case using any or all of those various data. This data assists the surgeons in determining where to resect and where to avoid (functional area of the brain) before they perform resective surgery. This data can be used to construct the clinicians' assumption of how the resection might be performed.

Although surgeons have a priori assumptions and information, in accordance with some embodiments of the invention, the surgeons can explore Granger Causality (GC) maps by changing some “parameters” before they make informed decision about the extent of resection or determine the margin of the resection. In accordance with some embodiments of the invention, the causality map obtained through the calculation can be modified by varying one or more of the following parameters, including the model order to adjust accuracy of the model and the normalized threshold values indicating causal strength or the number of ranked electrodes.

The GC maps can be tuned by adjusting the accuracy of the regression model by varying model order (i.e. the number of past time points of the data) used to construct the model. The GC analysis is done through regression modeling, and the accuracy of the model can affect causal interactions. In accordance with some embodiments, the model order can be varied from the lowest (which can be as low as 2) to maximum order which can be 10 or higher.

For example, in accordance with some embodiments, both the GC analysis (computation) process and the mapping (visualization) process can be integrated into a system that can analyze 5 min of electrocorticography (ECoG) data recorded in real time in the operating room (OR) with a particular model order (e.g., 2, 3, 4, 5, . . . 10, 11, 12, or more) and then visualize the GC map by presenting it to the clinical team, while the next 5 min of real time data is buffered and then the system can repeat the process until ECoG recording is completed.

In accordance with some embodiments, the time frame required for data buffering, computation and visualization process can be determined based on the speed of the system. Once the range of the time frame that the system can handle is set, the clinician can choose the size of the data segment to be processed for GC analysis and presented (e.g., mapped) for each time frame. In accordance with some embodiments, the time frame can be 5 min or shorter or longer than 5 min (for example: 1 min or 2 min or 3 min or 4 min or 5 min or 6 min or 7 min or 8 min or 9 min or 10 min or longer (depending on the recording status for each patient's case)). And different length time frames can be used for evaluating the same patient.

In accordance with some embodiments of the invention, a GC map can be made for each time frame and the surgeon (or the clinical team) can adjust the model order values in order to construct more useful and effective GC maps. In accordance with some embodiments of the invention, the system enables the surgeon or the clinical team to see the change in the GC maps when the model order is increased and decreased which can help the surgeon or the clinical team to determine the optimum boundary of the resection area.

In accordance with some embodiments of the invention, the system can vary the number of electrodes selected from the top rank. When one calculates Cartesian distance from “high causal nodes” set to clinically identified epileptogenic zone (EZ) set, one needs to determine how many electrodes from the top rank are considered as “high causal nodes” set. In accordance with some embodiments of the invention, the top five or top ten electrodes can be selected.

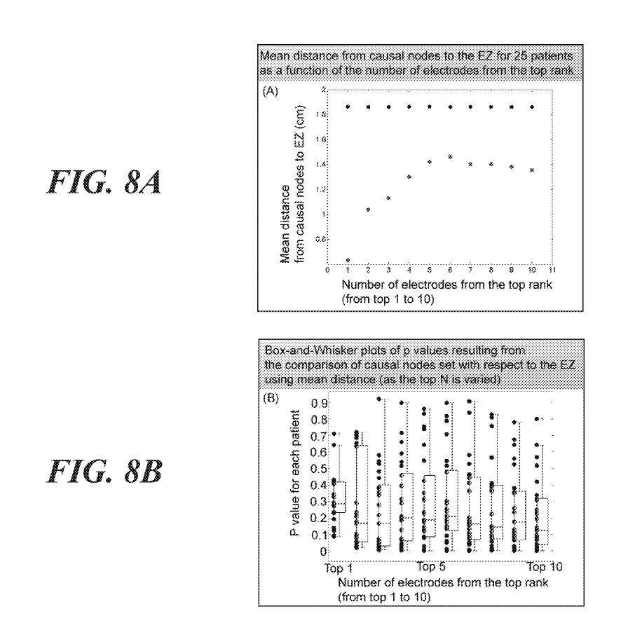

In accordance with some embodiments of the invention, the number of electrodes selected from the top rank can be varied in order to explore the changes in “distance” values. This adjustment based on the variation of the number of top N electrodes (N=1, 2, . . . , 10 or more) can be performed by surgeons when exploring GC maps in the OR, as desired (see FIG. 8A) to determine the extent of resection needed. Further, in accordance with some embodiments of the invention, the surgeon or the clinical team can modify and explore the resultant GC maps by adjusting this number of electrodes (which are the members of the “high causality set”).

In accordance with some embodiments, the surgeon or the clinical team can also vary the number of top N electrodes (e.g., where N=1, 2, to 10 or more) to examine the changes of p values (as outcome). This p value indicates “statistical comparison of distance from high causal nodes to the EZ with respect to the distance from randomly selected electrodes to the EZ set”. In the FIG. 8B, one can see Box-Whisker plot where p values for each patient (total 25 cases) are plotted as a function of the number of top N electrodes. FIG. 8B shows that this p value can change when the number of top N electrodes. So, GC maps can be explored by changing the number of electrodes (i.e. elements of the “high causal nodes set”) in terms of examining “p value” changes.

In accordance with some embodiments of the invention, the GC matrix can be changed by varying the normalized threshold values. The normalized threshold value based on causal strength can be varied to change and explore the distance values. In accordance with some embodiments of the invention, the system can score each electrode and rank it based on the score (e.g., the strength of causal interaction) when the system can set a different threshold value to determine the members of “high causality set” and as a result, the number of electrodes in the high causality set can be changed accordingly. Therefore, the threshold is a function of the number of top N electrodes in the high causality set.

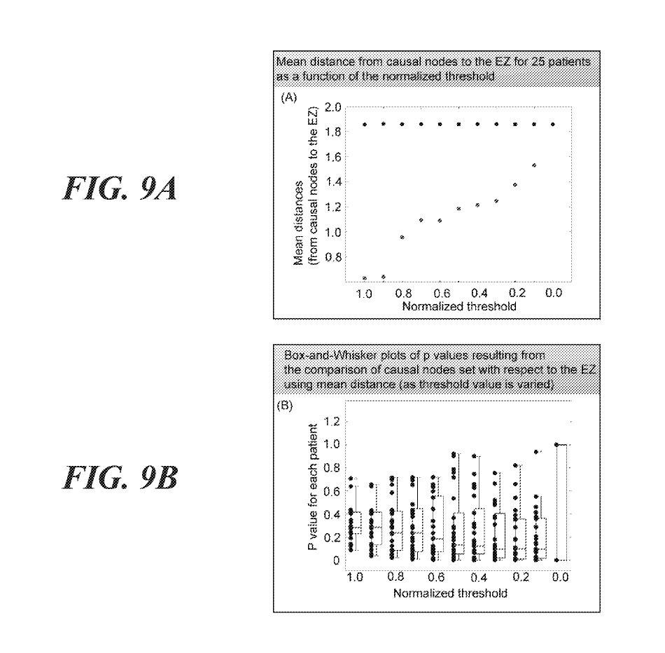

FIGS. 9A and 9B show similar plots as shown in FIGS. 8A and 8B. In FIG. 9, unlike FIG. 8, distance (FIG. 9A) and p values (FIG. 9B) change as the normalized threshold value (range between 0 and 1 with interval 0.1) varies.

In accordance with some embodiments of the invention, the system can adjust the resultant GC maps by changing the normalized threshold in terms of distance values and/or p values.

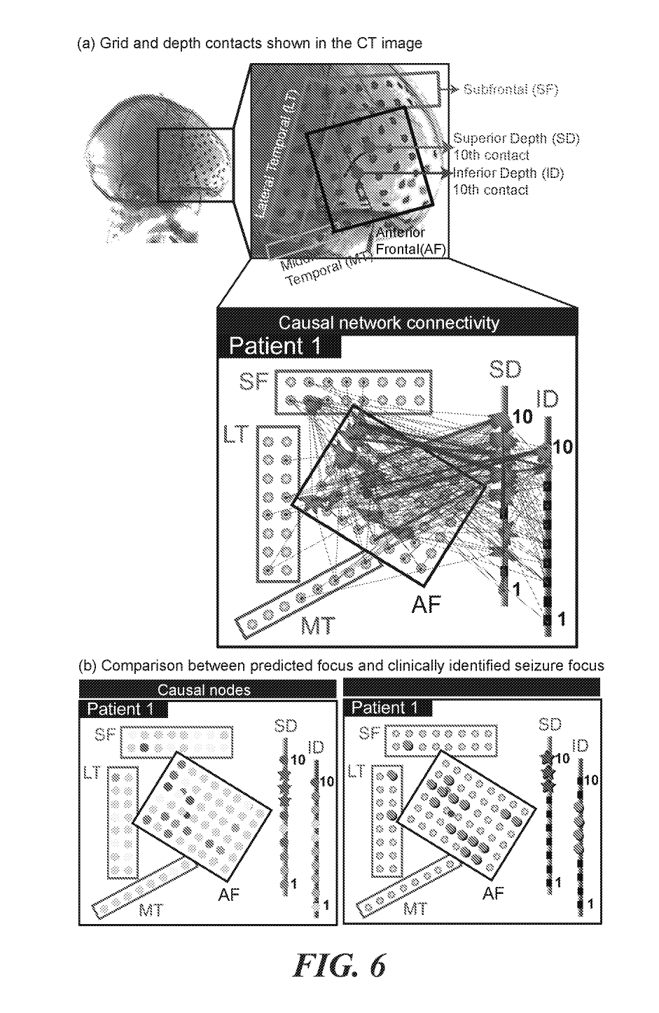

FIG. 6(a) shows an example of how arrays of grid and depth electrodes can be positioned on the brain according to an embodiment of the invention and the resulting map of the causality network connectivity determined according to some embodiments of the present invention.

FIG. 6(b) shows a comparison of the predicted causal nodes using interictal data and the seizure focus nodes identified by clinicians after observation of at least one seizure event for the same patient data.

In accordance with some embodiments of the invention, a patient specific recording grid of electrodes can be produced from prior 3D images and EEG data for the patient. In accordance with these embodiments, an image-derived, three dimensional, patient specific electrode grid (e.g., fabricated using 3D printing or machining) can be prepared for an individual patient. The patient specific electrode grid can be configured such that it positions within anatomical landmarks of the patient in one specific way. When the brain is exposed and the custom grid can be placed upon it, the location of each electrode will be already specified with respect to the brain in 3 dimensions. This enables the graphical representation of the electrode positions to be prepared before the surgery, and the visual information about the interactions among nodes can be more easily rendered upon this 3D model.

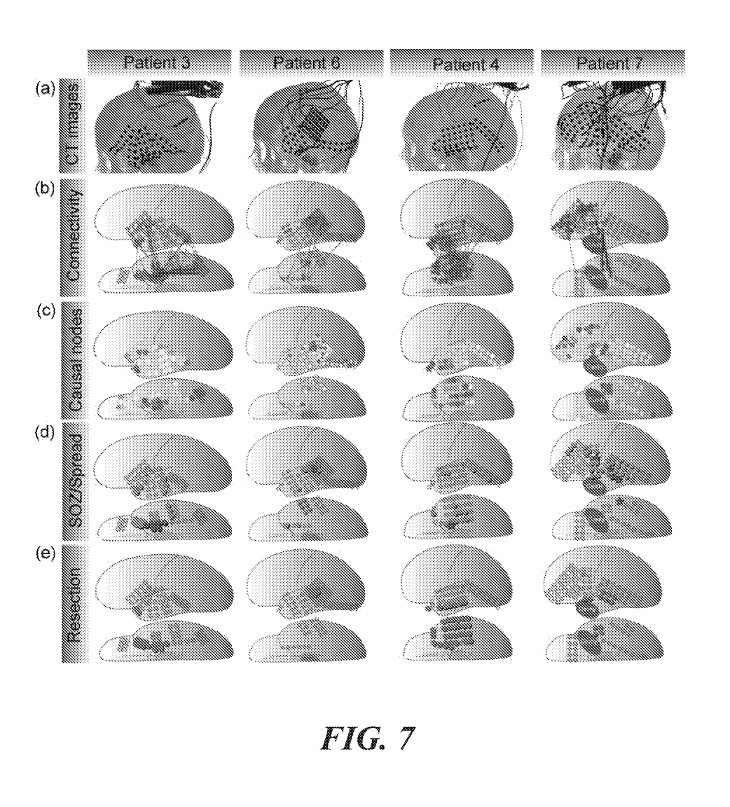

FIG. 7 shows diagrams of sample patient data showing (a) CT images of the brain showing grid placement; (b) schematic diagrams of the predicted node connectivity determined using interictal data according to the present invention; (c) schematic diagrams of the predicted causal nodes determined using interictal data according to the present invention; (d) schematic diagrams of the seizure onset zones and spread identified during at least one recorded seizure event; and (e) schematic diagrams of the resected locations.

As can be seen from the diagrams shown in FIG. 7, the methods and systems according to the invention can be used to identify and locate seizure onset zones from interictal data that closely correlate to seizure onset zones determined through the observance of a seizure event.

In accordance with some embodiments of the invention, the causality map information can be used to control a device or system that assists the clinical team in identifying one or more locations in the brain to be removed. For example, the array of electrodes can include a plurality of lights (LEDs) and the causality map information can be used to selectively illuminate the lights closest to the determined seizure foci locations. In some embodiments, different colors can be used to indicate rank or the most influential causal node locations. In accordance with some embodiments, the causality map information can be used to illuminate a beam of light or laser on the areas determined to contain the seizure focus. In accordance with some embodiments, the causality map information can be used to control or assist in the control of an automated or partially automated surgical system for locating and resecting the regions of seizure focus.

### REFERENCES

Each of the references cited herein are incorporated by reference in their entirety.
1. Anoruo E. Testing for Linear and Nonlinear Causality between Crude Oil Price Changes and Stock Market Returns. International Journal of Economic Sciences and Applied Research. 2011; 4 (3):75-92.
2. Granger C. Investigating causal relations by econometric models and cross-spectral methods, Econometrica. 1969; 37:424-438.
3. Granger C. Testing for causality: A personal viewpoint. Journal of Economic Dynamics and Control. 1980; 2(1):329-352.
4. Hiemstra C, Jones J D. Testing for Linear and Nonlinear Granger Causality in the Stock Price—Volume Relation. The Journal of Finance. 1994; 49(5):1639-1664
5. Liu Y, Bahadori M T. A Survey on Granger Causality: A Computational View http://www-bcfusc.edu/˜liu32/granger.pdf. 2012. (http://www-bcfusc.edu/˜liu32/granger.pdf).
6. D'Ausilio A, Badino L, Li Y, et al. Leadership in orchestra emerges from the causal relationships of movement kinematics. PLoS One. 2012; 7(5):e35757.
7. Ge T, Cui Y, Lin W, Kurths J, Liu C. Characterizing time series: when Granger causality triggers complex networks. New Journal of Physics. 2012; 14 (083028).
8. Marinazzo D, Pellicoro M, Stramaglia S. Kernel-Granger causality and the analysis of dynamical networks. Phys Rev E Stat Nonlin Soft Matter Phys. May 2008; 77(5 Pt 2):056215.
9. Krishna R, Li C T, Buchanan-Wollaston V. A temporal precedence based clustering method for gene expression microarray data. BMC Bioinformatics. 2010; 11:68.
10. Zou C, Ladroue C, Guo S, Feng J. Identifying interactions in the time and frequency domains in local and global networks—A Granger Causality Approach. BMC Bioinformatics. 2010; 11:337.
11. Detto M, Molini A, Katul G, Stoy P, Palmroth S, Baldocchi D. Causality and persistence in ecological systems: a nonparametric spectral granger causality approach. Am Nat. April 2012; 179(4):524-535.
12. Brovelli A, Ding M, Ledberg A, Chen Y, Nakamura R, Bressler S L. Beta oscillations in a large-scale sensorimotor cortical network: directional influences revealed by Granger causality. Proc Natl Acad Sci USA. Jun. 29 2004; 101(26):9849-9854.
13. Chen Y, Bressler S L, Ding M. Frequency decomposition of conditional Granger causality and application to multivariate neural field potential data. J Neurosci Methods. Jan. 30 2006; 150(2):228-237.
14. Ding M, Chen Y, Bressler S L. Granger Causality: Basic Theory and Application to Neuroscience. In: Schelter B, Winterhalder M, Timmer J, eds. Handbook of Time Series Analysis: Recent Theoretical Developments and Applications. 1 ed: Wiley-VCH; 2006.
15. Crone N E, Boatman D, Gordon B, Hao L. Induced electrocorticographic gamma activity during auditory perception. Brazier Award-winning article, 2001. Clin Neurophysiol. April 2001; 112(4):565-582.
16. Ludwig K A, Miriani R M, Langhals N B, Joseph M D, Anderson D J, Kipke D R. Using a common average reference to improve cortical neuron recordings from microelectrode arrays. J Neurophysiol. March 2009; 101(3):1679-1689.
17. McFarland D J, McCane L M, David S V, Wolpaw J R. Spatial filter selection for EEG-based communication. Electroencephalogr Clin Neurophysiol. September 1997; 103(3):386-394.
18. Ding M, Bressler S L, Yang W, Liang H. Short-window spectral analysis of cortical event-related potentials by adaptive multivariate autoregressive modeling: data preprocessing, model validation, and variability assessment. Biol Cybern. July 2000; 83(1):35-45.
19. Nagpaul P S. Time Series Analysis in WinIDAMS. New Delhi, India 2005.
20. Seth A K. A MATLAB toolbox for Granger causal connectivity analysis. J Neurosci Methods. Feb. 15 2010; 186(2):262-273.
21. Wilke C, Worrell G, He B. Graph analysis of epileptogenic networks in human partial epilepsy, Epilepsia. January 2011; 52(1):84-93.
22. Bressler S L, Seth A K. Wiener-Granger causality: a well established methodology. Neuroimage. Sep. 15 2011; 58(2):323-329.
23. Gow D W, Jr., Segawa J A, Ahlfors S P, Lin F H. Lexical influences on speech perception: a Granger causality analysis of MEG and EEG source estimates. Neuroimage. Nov. 15 2008; 43(3):614-623.
24. Simpson D M, Infantosi A F C, Junior J F C, Peixoto A J, Abrantes L Md. On the selection of autoregressive order for electroencephalographic (EEG) signals. Paper presented at: Circuits and Systems (Proceedings of the 38th Midwest Symposium); 13-16 Aug. 1995, 1995; Rio de Janeiro.
25. Palaniappan R. Towards optimal model order selection for autoregressive spectral analysis of mental tasks using genetic algorithm. International Journal of Computer Science and Network Security 2006; 6 (1A):153-162.
26. Akaike H. A new look at the statistical model identification. IEEE Transactions on Automatic Control. 1974; 19 (6):716-723.
27. Chen L L, Madhavan R, Rapoport B I, Anderson W S. Real-time brain oscillation detection and phase-locked stimulation using autoregressive spectral estimation and time-series forward prediction. IEEE Trans Biomed Eng. March 2013; 60(3):753-762.
28. Krusienski D J, McFarland D J, Wolpaw J R. An evaluation of autoregressive spectral estimation model order for brain-computer interface applications. Conf Proc IEEE Eng Med Biol Soc. 2006; 1:1323-1326.
29. McFarland D J, Wolpaw J R. Sensorimotor rhythm-based brain-computer interface (BCI): model order selection for autoregressive spectral analysis. J Neural Eng. June 2008; 5(2):155-162.
30. Durbin J, Watson G S. Testing for serial correlation in least squares regression. I. Biometrika. December 1950; 37(3-4):409-428.
31. Dagpunar J S. Simulation and Monte Carlo: With applications in finance and MCMC John Wiley & Sons Ltd; 2007.
32. Manly B F J. Randomization, Bootstrap and Monte Carlo Methods in Biology. 3 (Texts in Statistical Science Series) ed: Chapman and Hall/CRC; 2006.
33. Borror C M. Fleshing Things Out: Combining p-values to test for significance in a hypothesis. Quality Progress (STATISTICS ROUNDTABLE). September ed: www.qualityprogress.com; 2012
34. Elston R C. On Fisher's method of combining p-values. Biometrical Journal. 1991; 33:339.
35. Fisher R A. Statistical methods for research workers 11 ed. Edinburgh: Oliver and Boyd; 1950.
36. Jain A K, Murty M N, Flynn P J. Data clustering: a review. ACM Computing Surveys, 1999; 31(3):264.
37. Palla G, Farkas I J, Pollner P, Derényi I, Vicsek T. Fundamental statistical features and self-similar properties of tagged networks. New Journal of Physics. 2008; 10:123026.
38. Harih GaC, A. Open-source software in medical imaging: case-based study with interdisciplinary innovative product design. Acta Medico-Biotechnica. 2012; 5, (1):61.
39. Charles M. Epstein, Bhim M. Adhikari, Robert Gross, Jon Willie, and Mukesh Dhamala, “Application of high-frequency Granger causality to analysis of epileptic seizures and surgical decision making,” Epilepsia 2014 (November 4)
40. Chaudhary U J, Carmichael D W, Rodionov R, Thornton R C, Bartlett P, Vulliemoz S, Micallef C, McEvoy A W, Diehl B, Walker M C, Duncan J S, Lemieux L., Mapping preictal and ictal haemodynamic networks using video-electroencephalography and functional imaging, Brain, 2012, vol 135, p 3645-3663.
41. Lionel Barnett and Anil K. Seth, Behaviour of Granger causality under filtering: Theoretical invariance and practical application, Journal of Neuroscience Methods 201 2011 404-419.
42. Christoph Flamm, Andreas Graef, Susanne Pirker, Christoph Baumgartner, Manfred Deistler, Influence analysis for high-dimensional time series with an application to epileptic seizure onset zone detection, Journal of Neuroscience Methods 214, 2013, 80-90.
43. Schalk, Gerwin, Mellinger, Jürgen, General-Purpose Software for Brain-Computer Interface Research, Data Acquisition, Stimulus Presentation, and Brain Monitoring, A Practical Guide to Brain—Computer Interfacing with BCI2000, 2010, 22.
44. Sheng Lu, Ki Hwan Ju, and Ki H. Chon, “A New Algorithm for Linear and Nonlinear ARMA Model Parameter Estimation Using Affine Geometry”, IEEE Transactions on Biomedical Engineering, Vol. 48, No. 10, p 1116 (2001).
45. Rui Zou, Hengliang Wang, and Ki H. Chon, “A Robust Time-Varying Identification Algorithm Using Basis Functions”, Annals of Biomedical Engineering, Vol. 31, p 840 (2003).
46. Maria Peltola, Doctoral Thesis, “Electrical Status Epilepticus During Sleep Continuous Spikes and Waves During Sleep” (https://www.doria.fi/bitstream/handle/10024/98479/Maria%20Peltola%20DISS%202014.pdf?sequence=2)
47. Hamilton, J. D. (1994). Time series analysis. Princeton, N.J.: Princeton University Press.
48. P. Sundaram, W. M. Wells, R. V. Mulkern, M. Balasubramanian, E. J. Bubrick, and D. B. Orbach, Morphology of the eMRI Magnitude Response to Interictal Spikes: Timing, Amplitude and the Dip, Proc. Intl. Soc. Mag. Reson. Med., 19 (2011), p 1553

Other embodiments are within the scope and spirit of the invention. For example, due to the nature of software, functions described above can be implemented using software, hardware, firmware, hardwiring, or combinations of any of these. Features implementing functions may also be physically located at various positions, including being distributed such that portions of functions are implemented at different physical locations.

Further, while the description above refers to the invention, the description may include more than one invention.

What is claimed is:

1. A method for treating epileptic seizures comprising:
monitoring interictal EEG signals from a plurality of nodes associated with locations in the brain and recording the interictal EEG signals as interictal EEG data;
analyzing the interictal EEG data for two or more nodes to determine a causal influence of each of the two or more nodes;
ranking the nodes according to causal influence;
resecting a portion of the brain that corresponds to location of the causal nodes having a highest rank of causal influence based on the interictal EEG data.

2. The method according to claim 1 further comprising performing a craniotomy to expose a portion of the brain and placing one or more arrays of electrodes at node locations on the surface of the brain.

3. The method according to claim 2 further comprising positioning one or more electrodes below the surface of the brain.

4. The method according to claim 2 wherein the electrodes are arranged in a grid and the grid is configured in a specific pattern according to images of the brain of a specific patient.

5. The method according to claim 1 wherein the interictal EEG data is analyzed using a Granger Causality analysis to determine a measure of causal influence of two or more nodes.

6. The method according to claim 1 further comprising constructing a causal connectivity map that identifies causal nodes, causal connections and influenced nodes.

7. The method according to claim 1 further comprising controlling an indication system to indicate on a surface of the brain the location of the causal nodes having the highest rank of causal influence.

8. The method according to claim 1 further comprising controlling a surgical system to remove a portion of brain tissue at the location corresponding to the causal nodes having the highest rank of causal influence.

9. A system for treating epileptic seizures from interictal EEG data comprising:
a grid of electrodes for sensing interictal EEG signals, each electrode corresponding to a node location;
an interictal EEG signal collection system connected to the grid of electrodes for receiving the interictal EEG signals and storing interictal EEG data representative of the interictal EEG signals; and
the interictal EEG signal collection system including a computer processor and associated memory and having at least one program that analyzes the interictal EEG data for two or more node locations and determines based on the interictal EEG data a measure of causal influence of each node of the two or more nodes and an indication of causal connectivity between each of the nodes.

10. The system of claim 9 further comprising a database for storing the interictal EEG data.

11. The system of claim 9 further comprising an illumination system adapted to direct illumination on a portion of the brain corresponding to the location of at least one node having a high measure of causal influence.

12. The system of claim 9 further comprising a surgical robotic system connected to the interictal EEG signal collection system and receiving

13. The system of claim 12 wherein the surgical robotic system uses the data indicative of one or more node locations having a high measure of causal influence to determine parameters for a surgical procedure for removal of a portion of the brain.
